(19) United States
(12) Patent Application Publication
(10) Pub. No.: US 2025/0215387 A1
(43) Pub. Date: Jul. 3, 2025

(54) METHOD OF EFFICIENT CITRAMALATE PRODUCTION FROM BROWN MACROALGAE BIOMASS BY ENGINEERED VIBRIO SP. DHG

(71) Applicants: POSTECH RESEARCH AND BUSINESS DEVELOPMENT FOUNDATION, Pohang-si (KR); INHA UNIVERSITY RESEARCH AND BUSINESS FOUNDATION, Incheon (KR)

(72) Inventors: Gyoo Yeol JUNG, Pohang-si (KR); Hyun Gyu LIM, Incheon (KR); Sung Hwa WOO, Pohang-si (KR); Hye Kyung LEE, Pohang-si (KR)

(73) Assignees: POSTECH RESEARCH AND BUSINESS DEVELOPMENT FOUNDATION, Pohang-si (KR); INHA UNIVERSITY RESEARCH AND BUSINESS FOUNDATION, Incheon (KR)

(21) Appl. No.: 19/005,379

(22) Filed: Dec. 30, 2024

(30) Foreign Application Priority Data
Dec. 29, 2023 (KR) 10-2023-0196701

(51) Int. Cl. C12N 1/12; C12P 7/62; C12R 1/89

(52) U.S. Cl. CPC C12N 1/12; C12P 7/62; C12R 2001/89

(57) ABSTRACT

The present disclosure relates to efficient production of citramalate, which is a valuable industrial compound, from brown microalgae by using a transformed Vibrio sp. DHG strain. According to the present disclosure, it is possible to efficiently produce citramalate, which is used in various industrial fields, from brown microalgae-based raw materials by using a recombinant microorganism for producing citramalate. Therefore, the present disclosure not only introduces an innovative source for citramalate to advance the field, but also lays the foundation for extensive and efficient applications in bio-based industries.

### CROSS-REFERENCE TO RELATED APPLICATION

This present application claims the benefit of priority to Korean Patent Application No. 10-2023-0196701, entitled “METHOD OF EFFICIENT CITRAMALATE PRODUCTION FROM BROWN MACROALGAE BIOMASS BY ENGINEERED VIBRIO SP. DHG,” filed on Dec. 29, 2023, in the Korean Intellectual Property Office, the entire disclosure of which is incorporated herein by reference.

### INCORPORATION BY REFERENCE OF SEQUENCE LISTING

The content of the electronically submitted sequence listing, file name: Q305385_sequence listing as filed; size: 24,296 bytes; and date of creation: Dec. 27, 2024, filed herewith, is incorporated herein by reference in its entirety.

### FIELD

The present disclosure relates to efficient production of citramalate, which is a valuable industrial compound, from brown microalgae by using a transformed Vibrio sp. DHG strain.

### BACKGROUND

Seaweed has sufficient potential as promising alternative biomass sources for fossil fuels. In particular, brown microalgae exhibit higher productivity than conventional terrestrial plants and have advantages such as a high carbohydrate content, less competition with food resources, independence in terms of farmland and freshwater requirements, and ease of pretreatment. However, the use of alginate, a main carbohydrate of brown microalgae, has been difficult due to the limited metabolic ability of conventional industrial microorganisms. However, the surprising availability of alginate has been found by the discovery of a novel strain called Vibrio sp. DHG. In addition, the Vibrio sp. DHG strain may rapidly metabolize various carbon sources, including mannitol and glucose, which are abundant carbon sources in brown microalgae. In addition, easily available genetic engineering tools for the Vibrio sp. DHG strain have been established, which may be utilized in various fields of production of high value-added compounds using microorganisms.

Citramalate is a valuable compound used in various fields, such as food, cosmetics, pharmaceuticals, and plastics industries. In particular, the citramalate is used as a precursor of methyl methacrylate, which is a monomer of polymethyl methacrylate. The annual global market scale of methyl methacrylate is estimated at approximately 2.2 million tons, but conventional synthetic methods are difficult due to problems such as treatment of sulfuric acid and toxic cyanide. Therefore, a combined method that combines microbial production of citramalate and subsequent conversion to methyl methacrylate using a chemical catalyst is emerging as an alternative. Citramalate synthase (CimA) derived from Methanocaldococcus jannaschii or Geobacter sulfurreducens synthesizes citramalate by catalytic coupling of pyruvate and acetyl CoA. Active research has been conducted to improve citramalate production from microorganisms by utilizing various metabolic engineering strategies. However, most previous studies have been limited in the utilization of raw materials because of most conversion from glucose.

### SUMMARY

The present disclosure has been made to solve the problems of the related art, and an aspect of the present disclosure is to provide a transformed Vibrio sp. DHG strain capable of efficiently synthesizing citramalate using brown microalgae-based raw materials and a method for producing citramalate using the same.

In order to solve the aforementioned problem, the present disclosure provides a recombinant Vibrio sp. DHG strain into which an expression cassette including a cimA3.7 gene represented by SEQ ID NO: 1 is introduced.

The expression cassette may include a Ptac promoter represented by SEQ ID NO: 3 and a 5′ UTR represented by SEQ ID NO: 4.

The expression cassette may be introduced into a plasmid pACYC of a P15A replication origin.

The expression cassette may be introduced into a plasmid pJUMP24-1A of a pRO1600/ColE1 mixed replication origin.

The strain may be strains from which at least one gene or operon selected from the group consisting of a 1dhA gene represented by SEQ ID NO: 5, a frdABCD operon represented by SEQ ID NOs: 6 to 9, an ackA-pta operon represented by SEQ ID NOs: 10 and 11, and a leuCD operon represented by SEQ ID NOs: 12 and 13 is deleted.

The strain may be strains from which a ldhA gene represented by SEQ ID NO: 5, a frdABCD operon represented by SEQ ID NOs: 6 to 9, an ackA-pta operon represented by SEQ ID NOs: 10 and 11, and a leuCD operon represented by SEQ ID NOs: 12 and 13 are deleted.

Further, the present disclosure provides a method for producing citramalate including culturing the strain described above.

In the culturing, the carbon source of the medium may include alginate and mannitol.

In the culturing, the carbon source of the medium may be brown microalgae. According to the present disclosure, it is possible to efficiently produce citramalate,

which is used in various industrial fields, from brown microalgae-based raw materials by using a recombinant microorganism for producing citramalate. Therefore, the present disclosure not only introduces an innovative source for citramalate to advance the field, but also lays the foundation for extensive and efficient applications in bio-based industries.

### BRIEF DESCRIPTION OF THE DRAWINGS

The above and other aspects, features, and advantages of the present disclosure will become apparent from the detailed description of the following aspects in conjunction with the accompanying drawings, in which:

FIG. 1 is a diagram showing introduction of citramalate synthase of a Vibrio sp. DHG strain according to the present disclosure and a citramalate production pathway from alginate and mannitol, which are brown microalgae-based raw materials;

FIG. 2 is a diagram showing growth curves of VLC, VHC, and VXHC strains in a minimal medium containing alginate and mannitol;

FIG. 3 is a diagram showing growth curves (A and B) of the VXHC strain in minimal mediums containing various ratios of alginate and mannitol and results (C) of analyzing the production of citramalate and by-products of the VXHC strain when the ratio of alginate and mannitol is 1:1, 1:2, and 2:1; and

FIG. 4 is a diagram comparing carbon source analysis and citramalate production of brown microalgae according to a brown microalgae type of a VXHC strain.

### DETAILED DESCRIPTION

Hereinafter, the present disclosure will be described in detail.

The present inventors have completed the invention relating to a transformed Vibrio sp. DHG strain having a citramalate producing ability into which a cimA3.7 gene represented by a nucleotide sequence of SEQ ID NO: 1 is introduced, and a method for producing citramalate including culturing the transformed Vibrio sp. DHG strain.

Accordingly, the present disclosure provides a recombinant Vibrio sp. DHG strain into which an expression cassette including a cimA3.7 gene represented by SEQ ID NO: 1 is introduced.

The expression cassette may include a Ptac promoter represented by SEQ ID NO: 3 and a 5′ UTR represented by SEQ ID NO: 4.

The expression cassette may be introduced into plasmid pACYC of a P15A replication origin.

The expression cassette may be introduced into plasmid pJUMP24-1A of a pRO1600/ColE1 mixed replication origin.

The strain may be strains from which at least one gene or operon selected from the group consisting of a ldhA gene represented by SEQ ID NO: 5, a frdABCD operon represented by SEQ ID NOs: 6 to 9, an ackA-pta operon represented by SEQ ID NOs: 10 and 11, and a leuCD operon represented by SEQ ID NOs: 12 and 13 is deleted.

The strain may be strains from which a ldhA gene represented by SEQ ID NO: 5, an frdABCD operon represented by SEQ ID NOs: 6 to 9, an ackA-pta operon represented by SEQ ID NOs: 10 and 11, and a leuCD operon represented by SEQ ID NOs: 12 and 13 are deleted.

Further, the present disclosure provides a method for producing citramalate including culturing the strain described above.

In the culturing, the carbon source of the medium may include alginate and mannitol.

In the culturing, the carbon source of the medium may be brown microalgae.

Unless otherwise defined, the technical and scientific terms used herein have meanings commonly understood by those skilled in the art to which the present disclosure pertains. In addition, repeated descriptions of the same technical configuration and function as in the related art will be omitted.

According to an aspect of the present disclosure, the present disclosure provides a transformed Vibrio sp. DHG strain having a citramalate producing ability into which a cimA3.7 gene represented by a nucleotide sequence of SEQ ID NO: 1 is introduced.

The Vibrio sp. DHG strain used in the present disclosure has a high-performance carbon source metabolic pathway. The Vibrio sp. DHG strain was isolated and identified from seaweed sludge and was deposited with the Korean Collection for Type Cultures under the Korea Research Institute of Bioscience and Biotechnology on Apr. 6, 2017, and was assigned the deposit number KCTC13239BP. The Vibrio sp. DHG strain has a much higher growth rate than microorganisms such as E. coli in minimal media/nutrient media, and shows resistance to high initial sugar/salt concentrations.

In the present disclosure, the “carbon source” means a carbon compound that is absorbed into a living organism and used as a biocomponent carbon, and in the culturing the strain, the carbon source is used to clarify a physiological relationship with a nutrient source, and accordingly, the isolation and growth characteristics of the strain are confirmed.

In a specific example of the present disclosure, the Vibrio sp. DHG strain includes a 16S rDNA gene represented by a nucleotide sequence of SEQ ID NO: 2.

In the present disclosure, the “gene” should be considered in the broadest sense and may encode a structural protein or regulatory protein. In this case, the regulatory protein includes a transcription factor, a heat shock protein, or a protein involved in DNA/RNA replication, transcription, and/or translation. In the present disclosure, the target gene to be suppressed in expression may exist as an extrachromosomal component.

In the present disclosure, citramalate is an organic acid consisting of five carbons and is used as precursors of other compounds and the like due to its structural characteristics.

The transformed Vibrio sp. DHG strain into which the cimA3.7 gene is introduced is heterologously expressed to establish a citramalate isomerization pathway. In addition, the transformed Vibrio sp. DHG strain may include a promoter Ptac (SEQ ID NO: 3) verified through linear studies and an optimized 5′ UTR sequence (SEQ ID NO: 4) generated through a UTR designer for stable and constant expression of the cimA3.7 gene.

In a specific embodiment of the present disclosure, the Vibrio sp. DHG strain introduced with a plasmid pACYC of a P15A replication origin including the cimA3.7 gene cassette is named as VLC.

In a specific embodiment of the present disclosure, the Vibrio sp. DHG strain introduced with a plasmid pJUMP24-1A of a pRO1600/ColE1 mixed replication origin including the cimA3.7 gene cassette is named as VHC.

In a specific embodiment of the present disclosure, the strain may be strains from which at least one gene selected from the group consisting of an ldhA gene encoding lactate dehydrogenase represented by a nucleotide sequence of SEQ ID NO: 5, a frdABCD operon encoding fumarate reductase represented by nucleotide sequences of SEQ ID NOs: 6 to 9, an ackA-pta operon encoding acetate kinase and phosphotransacetylase genes represented by the nucleotide sequences of SEQ ID NOs: 10 and 11, and a leuCD operon encoding isopropylmalate isomerase represented by nucleotide sequences of SEQ ID NOs: 12 and 13 are deleted, and more preferably, the genes ldhA, frdA, frdB, frdC, frdD, ackA, pta, leuC and leuD are all deleted. The strain from which the genes ldhA, frdA, frdB, frdC, frdD, ackA, pta, leuC, and leuD are all deleted was named VXC. In order to efficiently produce citramalate, in the VXC strain, the related genes ldhA, frdABCD, ackA-pta, and leuCD are deleted to prevent a by-product production pathway and the loss of citramalate.

In addition, variants of the above-mentioned nucleotide sequences are included within the scope of the present disclosure. Specifically, the variants have sequence homology of 70% or more, more preferably 80% or more, even more preferably 90% or more, and most preferably 95% or more with the above-mentioned nucleotide sequences, and mean sequences that exhibits substantially the same physiological activity as the above-mentioned nucleotide sequences. The “% of sequence homology” with the polynucleotide is confirmed by comparing two optimally aligned sequences with a comparison region, and a part of a polynucleotide sequence in the comparison region may include addition or deletion (i.e., gap) compared to a reference sequence (without including addition or deletion) for an optimal alignment of the two sequences.

The Vibrio sp. DHG strain, in which the plasmid pJUMP24-1A of the pRO1600/ColE1 mixed replication origin including the cimA3.7 gene cassette is introduced into the VXC strain, was named VXHC.

It was confirmed through the experiment that the transformed Vibrio sp. DHG strain according to the present disclosure produced 8.6 g/L of citramalate in a minimal medium containing alginate and mannitol for 48 hours. In addition, it was confirmed that citramalate was produced in various brown microalgae, and particularly, it was confirmed that up to 8.7 g/L of citramalate was produced in kelp, which is one of the brown microalgae species. The production of citramalate using the transformed Vibrio sp. DHG strain corresponds to the productivity of 0.18 g/L/h, which is a higher value than the results of previous studies conducted using E. coli. Therefore, the transformed Vibrio sp. DHG strain according to the present disclosure may be utilized in various fields of high value-added production as well as citramalate using brown microalgae.

In the present disclosure, a 5′ untranslated region (5′ UTR) is an untranslated region at the 5′ end and 3′ end of mRNA, and generally, the 5′ untranslated region of mRNA performs various functions including participating in the regulation of mRNA translation efficiency during a gene expression process. It has been reported that a nucleotide sequence of the 5′ UTR present in a 5′ upper portion of a translation initiation codon affects the efficiency of the translation step, and the length of the 5′ UTR consists of 100 bases or longer nucleotides, and the length of a 3′ UTR consists of several kilobases longer. In addition, it has been reported results of studies on sequences belonging to the 5′ UTR, which may be referred to as a ribosome binding site sequence even in eukaryotes, not at a fixed position such as a Shine-Dalgarno sequence, which is known as a ribosome binding site sequence located in a 5′ UTR in prokaryotes.

In the present disclosure, the expression cassette refers to a unit cassette that includes a promoter and a gene encoding a target protein and may be expressed to produce the target protein operably linked to the downstream of the promoter. Various factors capable of helping the efficient production of the target protein may be included inside or outside such an expression cassette. Specifically, in the target protein expression cassette, the gene encoding the target protein may be operably linked to the downstream of the promoter sequence.

The ‘operably linked’ means that the gene sequence and the promoter sequence are functionally linked to each other so that a nucleic acid sequence having the promoter activity of the present disclosure initiates and mediates the transcription of the gene encoding the target protein. The operable linkage may be prepared using genetic recombination techniques known in the art, and site-specific DNA cleavage and linkage may be prepared using cleavage and linkage enzymes in the art, but are not limited thereto. That is, the ‘recombinant gene expression cassette’ may be inserted into a chromosome of a host cell and used to prepare a recombinant microorganism, and it is obvious for those skilled in the art to which the present disclosure pertains to have the same effect as the case of introducing the recombinant vector into the host cell as described above even though the recombinant gene expression cassette is inserted into the genomic chromosome of the host cell. As a method of inserting the recombinant gene expression cassette into the chromosome of the host cell, conventionally known genetic engineering methods may be used. As an example, the method includes methods of using a retrovirus vector, an adenovirus vector, an adeno-associated virus vector, a herpes simplex virus vector, a poxvirus vector, a lentiviral vector, or a non-viral vector.

In the present disclosure, the “promoter” refers to an untranslated nucleic acid sequence upstream of a coding region that includes a binding site for polymerase and has transcription initiation activity into mRNA of a gene downstream of the promoter, i.e., a DNA region to which the polymerase binds to initiate transcription of a gene. The promoter may be located at a 5′ site of the mRNA transcription initiation region.

In the present disclosure, the “vector” refers to a genetic construct including a nucleotide sequence of a gene operably linked to a suitable regulatory sequence so as to express a target gene in a suitable host. The regulatory sequence may include a promoter capable of initiating transcription, any operator sequence for regulating such transcription, and sequences for regulating termination of transcription and translation. The vector of the present disclosure is not particularly limited as long as the vector is replicable in cells, and may use any vector known in the art, for example, a plasmid, a cosmid, a phage particle, or a viral vector.

In the present disclosure, when a coding gene of a target polypeptide to be expressed is operably linked, the recombinant vector may be used as an expression vector of a target polypeptide capable of expressing the target polypeptide with high efficiency in a suitable host cell, and the recombinant vector may be expressed in a host cell. The host cell may preferably be a eukaryotic cell, and expression regulatory sequences such as a promoter, a terminator, and an enhancer, sequences for membrane targeting or secretion, and the like are appropriately selected according to a type of host cell and may be variously combined depending on a purpose.

In the present disclosure, the recombinant microorganism refers to a microorganism transformed with the recombinant vector of the present disclosure. In the present disclosure, the ‘transformation’ means introducing a vector including the promoter according to the present disclosure or further including the gene encoding the target protein into a host cell. In addition, the gene encoding the transformed target protein may be inserted and located into the chromosome of the host cell or located outside the chromosome, as long as the gene may be expressed in the host cell.

According to yet another aspect of the present disclosure, the present disclosure provides a method for producing citramalate including culturing a recombinant microorganism for producing the citramalate.

The medium and other culture conditions used for culturing the microorganism of the present disclosure may be used with any medium to be used for culturing conventional Vibrio sp. microorganisms, but need to suitably satisfy the requirements of the microorganism of the present disclosure. Preferably, the microorganism of the present disclosure is cultured in a conventional medium containing suitable carbon sources, nitrogen sources, amino acids, vitamins, etc. under aerobic conditions while controlling temperature, pH, and the like.

In a preferred embodiment of the present disclosure, the medium may include sugars or sugar alcohols as carbon sources, more specifically at least one selected from the group consisting of glucose, mannitol, sucrose, arabinose, galactose, glycerol, xylose, mannose, fructose, lactose, maltose, sucrose, alginate, cellulose, dextrin, glycogen, hyaluronic acid, lentinan, zymosan, chitosan, glucan, lignin and pectin, preferably at least one selected from the group consisting of glucose, mannitol, alginate, sucrose, arabinose, galactose and glycerol, but is not limited thereto. As the inorganic compound, sodium chloride, calcium chloride, iron chloride, magnesium sulfate, iron sulfate, manganese sulfate, calcium carbonate, and the like may be used, and in addition, amino acids, vitamins, suitable precursors, and the like may be included. These media or precursors may be added to a culture medium in a batch or continuous culture.

During the culture, compounds such as ammonium hydroxide, potassium hydroxide, ammonia, phosphoric acid, and sulfuric acid are added to the culture medium by a proper method to adjust the pH of the culture medium. In addition, during the culture, the generation of bubbles may be suppressed by using an anti-foaming agent such as fatty acid polyglycol ester. Further, in order to maintain an aerobic condition of the culture medium, oxygen or oxygen-containing gases may be injected into the culture medium, or in order to maintain anaerobic and aerobic conditions, nitrogen, hydrogen, or carbon dioxide gas may be injected without injection of gases.

The temperature of the culture medium may be set usually 27° C. to 37° C., preferably 30° C. to 35° C. The culture period may be continued until a desired production of useful materials is obtained, preferably for 10 to 100 hours.

The method may further include purifying or recovering the produced citramalate in the culturing step of the present disclosure, and a method for recovering the citramalate from the microorganism or culture medium may be used with methods known in the art, such as centrifugation, filtration, anion exchange chromatography, crystallization, and HPLC, but is not limited to these examples.

The recovering step may include a purification process, and those skilled in the art may select and utilize various known purification processes as needed.

Duplicated contents are omitted in consideration of the complexity of the present specification, and terms not defined otherwise in the present specification have the meanings commonly used in the art to which the present disclosure pertains.

Hereinafter, the present disclosure will be described in more detail with reference to specific Examples. The following Examples describe preferred specific examples of the present disclosure, and the scope of the present disclosure is not limited by the matters described in the following Examples.

### EXAMPLES

### Example 1. Construction of Citramalate Production Isomerization Pathway in Vibrio sp. DHG Strain

Through genome analysis of a Vibrio sp. DHG strain (Lim et al., 2019, KCTC13239BP), it was confirmed whether the Vibrio sp. DHG strain naturally had a pathway required for citramalate production. As a result, it was confirmed that the Vibrio sp. DHG strain did not have a gene set for completing the citramalate production pathway.

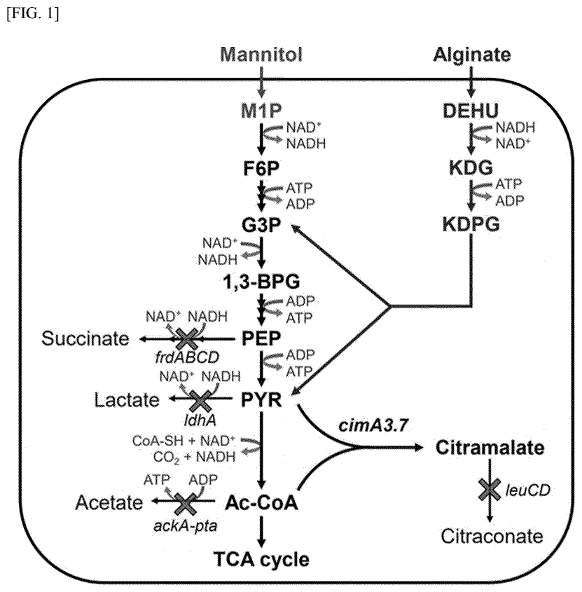

To construct the citramalate production isomerization pathway, the cimA3.7 gene (SEQ ID NO: 1) was heterologously expressed in the Vibrio sp. DHG strain as shown in FIG. 1. A promoter Ptac (SEQ ID NO: 3) verified through linear studies and an optimized 5′ UTR sequence (SEQ ID NO: 4) generated through a UTR designer were used for stable and constant expression of a cimA3.7 gene.

A Vibrio sp. DHG strain introduced with a plasmid pACYC of a P15A replication origin including the cimA3.7 gene cassette was named VLC.

A Vibrio sp. DHG strain introduced with a plasmid pJUMP24-1A of a pRO1600/ColE1 mixed replication origin including the cimA3.7 gene cassette was named VHC.

The information about detailed genotypes and plasmids of strains used thereafter was shown in Table 1.

TABLE 1

| Name | Description | Source |
|---|---|---|
| Strains | | |
| E. coli | A host for the cloning of plasmids with R6Kp15A and | Invitrogen |
| M [symbol] -T [symbol] | pRO1600C [symbol] hybrid origin | |
| E. coli EC200D | Parental strain for E. coli EC100D [symbol] -116 pRSF_c [symbol] A | Epicentre |
| [symbol] -116 | | |
| E. coli EC100D | A host for the cloning of plasmids with R6K origin | Present invention |
| [symbol] -116 | | |
| pRSF_ [symbol] A | | |
| E. coli S17-1 | Parental strain for E. coli S17-1 [symbol] | ATCC 47055 |
| E. coli S17-1 [symbol] | Parental strain for donor strain | Present invention |
| ED_ [symbol] | E.coli S17-1 [symbol] R6K_ [symbol] _ [symbol] | Present invention |
| ED_ [symbol] CD | E.coli S17-1 [symbol] R6K_ [symbol] B_ [symbol] CD | Present invention |
| VDHG | Wildtype [symbol] | (Li [symbol] et al., |
| | | 2019) |
| VDHG300 | VDHG [symbol] BCD | (Li [symbol] et al., |
| | | 2019) |
| VX | VDHG [symbol] BCD [symbol] CD | Present invention |
| VLC | VDHGpACYC_c [symbol] A | Present invention |
| VHC | VDHGpJUMP_c [symbol] A | Present invention |
| VXHC | VXpJUMP_c [symbol] A | Present invention |
| Plasmids | | |
| pACYC_Duet | p15A, L [symbol] , C [symbol] | Novagen |
| pACYA | p15A, L [symbol] , A [symbol] | (Li [symbol] et al., |
| | | 2019) |
| pRSF_Duet | RSF1030, L [symbol] , K [symbol] | Novagen |
| pJUMP24- [symbol] A( [symbol] PP) | pRO1600C [symbol] , K [symbol] | Val [symbol] -O |
| | | [symbol] and |
| | | [symbol] 2021) |
| pRSF_FLP | RSF1030 P [symbol] _ [symbol] UTR_ [symbol] | (Li [symbol] et al., |
| | | 2019) |
| pACYA_FLP | p15A, L [symbol] , A [symbol] R, PVP13_sy [symbol] UTR_f [symbol] C | Present invention |
| pSAM_AmC | Source of R6K and [symbol] nT | Novagen |
| p11-L [symbol] Y [symbol] | Source of A [symbol] C_P [symbol] _c [symbol] B_ [symbol] B terminator | Addgene |
| FRT [symbol] _ [symbol] | Source of C [symbol] [symbol] ed by FRT sequence | (Li [symbol] et al., |
| | | 2019) |
| R6K_ [symbol] B_Back | R6K, [symbol] T, CmR, A [symbol] C, P [symbol] _c [symbol] B terminator | Present invention |
| bone | | |
| pRSF_ccdA | RSF1030 Km [symbol] [symbol] 23101_c [symbol] A_BB [symbol] _B1001 terminator | Present invention |
| R6K_ [symbol] B_ [symbol] A- | R6K, [symbol] T, A [symbol] C, P [symbol] _c [symbol] B_ [symbol] B terminator C [symbol] ed by FRT, | Present invention |
| p [symbol] | [symbol] ed by homology for deletion of [symbol] | |
| R6K_ [symbol] B_ [symbol] CD | R6K, [symbol] T, A [symbol] C, P [symbol] , [symbol] B terminator C [symbol] ed by FRT, | Present invention |
| | [symbol] ed by homology for deletion of [symbol] CD | |
| pACYC_ [symbol] mA3.7 | p15A, CmR, P [symbol] _sys [symbol] -UTR_ [symbol] A3.7_BB [symbol] _B1006 terminator | Present invention |
| pJUMP_ [symbol] mA3.7 | pRO1600 C [symbol] , [symbol] mR, P [symbol] _sys | Present invention |
| | [symbol] _UTR_c [symbol] A3.7_BB [symbol] _B1006 terminator | |

[symbol]

indicates data missing or illegible when filed

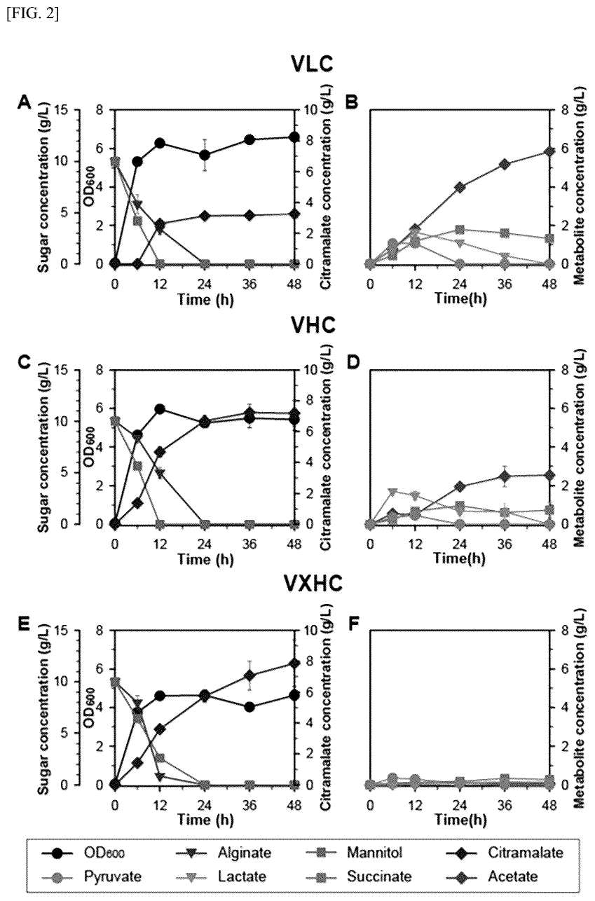

The constructed VLC and VHC strains were cultured in a medium containing a mixture of alginate and mannitol at a ratio of 1:1 for 48 hours, and OD₆₀₀, consumed carbon sources, citramalate, and by-product production were analyzed. The results of analyzing OD₆₀₀, consumed carbon sources, and citramalate of VLC were shown in A of FIG. 2, and the results of analyzing by-product production were shown in B of FIG. 2. In addition, the results of analyzing OD₆₀₀, consumed carbon sources, and citramalate of VHC were shown in C of FIG. 2, and the results of analyzing by-product production were shown in D of FIG. 2.

As shown in A and C of FIG. 2, the VLC and VHC strains produced 3.3 g/L and 7.2 g/L of citramalate, respectively, to prove the functional expression of the cimA3.7 gene, and it was confirmed that the expression amplification of cimA3.7 increased the production of citramalate by 2.2 times.

In experiments to be described below, routine cell culturing at a flask scale was performed by picking colonies from an LB or LBv2 medium (10 g/L tryptone, 5 g/L yeast extract, 21.92 g/L NaCl, 0.3 g/L KCI, and 2.2 g/L MgCl₂) agar plate and inoculating the colonies in a 15 mL test tube containing 3 mL of a M9 or modified minimal medium for citramalate production (5 g/L (NH₄)₂SO₄, 30 g/L NaCl, 10.7 g/L KH₂PO₄, 5.2 g/L KH₂PO₄, 0.5 g/L MgSO₄·7H₂O, 4.5 mg/L thiamine·HCl, 200 mg/L citric acid, 200 mg/L L-leucine, 5 g/L yeast extract, and 2 mL/L trace metal solution). The corresponding culture solution was cultured overnight, and then re-inoculated into a new medium at OD₆₀₀ of 0.05. When OD₆₀₀ reached 1.0, the culture solution was re-inoculated into a 350 mL Erlenmeyer flask containing 25 mL of a modified minimal medium at OD₆₀₀ of 0.05. The culturing was performed at 30° C. and 200 rpm in a rotary shaker. Appropriate antibiotics were added, and all cell culturing was performed in triplicate.

### Example 2. Production of Recombinant Microbial Strain With Further Improved Citramalate Production Ability

In Example 1, when citramalate production was confirmed, a Vibrio sp. DHG strain was further improved so that the citramalate production pathway was shown in FIG. 1. Specifically, in order to efficiently produce citramalate, a VDHG300 strain, from which IdhA gene and frdABCD genes were deleted in previous studies, was used. The ldhA gene was represented by a nucleotide sequence of SEQ ID NO: 5, and frdA, frdB, frdC, and frdD were represented by nucleotide sequences of SEQ ID NOs: 6 to 9. Additionally, an ackA gene and a pta gene were deleted to block the by-product production pathway. The ackA gene was represented by a nucleotide sequence of SEQ ID NO: 10, and the pta gene was represented by a nucleotide sequence of SEQ ID NO: 11. Additionally, a leuC gene and a leuD gene were deleted to prevent the loss of citramalate. The leuC gene was represented by a nucleotide sequence of SEQ ID NO: 12, and the leuD gene was represented by a nucleotide sequence of SEQ ID NO: 13. As described above, the strain from which the ackA-pta and leuCD genes were deleted was named VX by further improving the VDHG300 strain, from which ldhA and frdABCD were deleted. The Vibrio sp. DHG strain, in which the plasmid pJUMP24-1A of the pRO1600/ColE1 mixed replication origin including the cimA3.7 gene cassette was introduced into the VX strain, was named VXHC.

The constructed VXHC strain was cultured in a medium containing a mixture of alginate and mannitol at a ratio of 1:1 for 48 hours, and OD₆₀₀, consumed carbon sources, citramalate, and by-product production were analyzed. In addition, the results of analyzing OD₆₀₀, consumed carbon sources, and citramalate were shown in E of FIG. 2, and the results of analyzing by-product production were shown in F of FIG. 2.

As shown in E of FIG. 2, 7.9 g/L of citramalate was produced for 48 hours, which was confirmed to have a 10% increase compared to conventional VHC strains.

### Example 3. Demonstration of Efficient Conversion Method of Brown Microalgae-Based Citramalate Using Transformed Strain

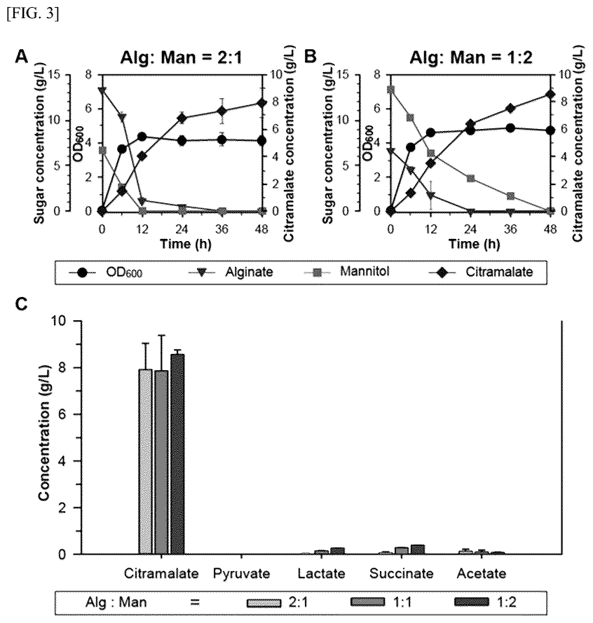

A VXHC strain, which was improved to efficiently produce citramalate by rapidly utilizing brown algae-derived carbon sources, was cultured in media with various changed alginate and mannitol compositions. The improved VXHC strain was cultured in media containing the mixture of alginate and mannitol at a ratio of 2:1 and 1:2, and the OD₆₀₀, consumed carbon sources, citramalate, and by-product production were analyzed. The results of analyzing OD₆₀₀, consumed carbon sources, and citramalate were shown in A of FIG. 2 when VXHC was cultured in a medium containing a mixture of alginate and mannitol at a ratio of 2:1. The results of analyzing OD₆₀₀, consumed carbon sources, and citramalate were shown in B of FIG. 3 when VXHC was cultured in a medium containing a mixture of alginate and mannitol at a ratio of 1:2. The results of comparing the production of citramalate and by-products were shown in C of FIG. 3.

As shown in A and B of FIG. 3, when the ratio of alginate and mannitol was changed to 2:1 and 1:2, citramalate was produced at 7.9 g/L and 8.6 g/L, respectively, which was similar to that when added at 1:1. When the ratio of mannitol was increased, the accumulation of by-products was slightly increased, as shown in C of FIG. 3. It was confirmed that the production of citramalate was caused by the lack of dependence on redox cofactors. The results emphasized the stable citramalate production possibility from brown microalgae-based supply sources with various carbon source compositions.

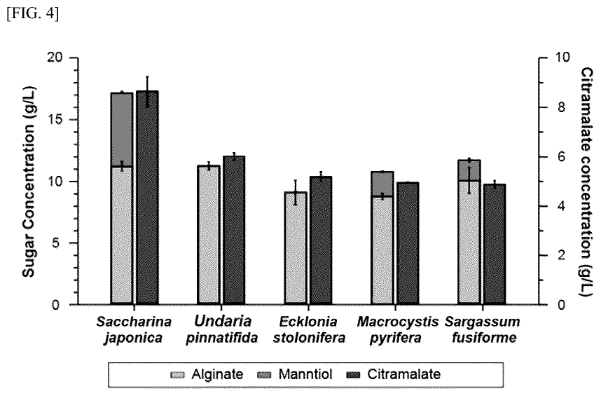

The VXHC strain, which was improved to efficiently produce citramalate by rapidly utilizing brown microalgae-derived carbon sources, was cultured in media containing five well-known brown microalgae species, Saccharina japonica, Undaria pinnatifida, Ecklonia Stolonifera, Macrocystis pyrifera, and Sargassum fusiforme, respectively, to produce citramalate, and the results were shown in FIG. 4. This achievement was critical in that citramalate production using raw biomass, especially brown microalgae was first demonstrated.

As shown in FIG. 4, 8.7 g/L of citramalate was produced in Saccharina japonica for 72 hours, which corresponded to the productivity of 0.12 g/L/h. This is because Saccharina japonica has the highest carbohydrate content among brown microalgae. This suggests that the applicability of the strain may be greatly expanded by optimizing the selection of brown microalgae with a high carbohydrate content and improving cultivation techniques. The results not only advance the corresponding field, but also lay the foundation for extensive and efficient applications in bio-based industries by introducing a new source of citramalate.

1. A recombinant Vibrio sp. DHG strain into which an expression cassette including a cimA3.7 gene represented by SEQ ID NO: 1 is introduced.

2. The strain of claim 1, wherein the expression cassette includes a Ptac promoter represented by SEQ ID NO: 3 and a 5′ UTR represented by SEQ ID NO: 4.

3. The strain of claim 2, wherein the expression cassette is introduced into a plasmid pACYC of a P15A replication origin.

4. The strain of claim 2, wherein the expression cassette is introduced into a plasmid pJUMP24-1A of a pRO1600/ColE1 mixed replication origin.

5. The strain of claim 4, wherein the strain is a strain from which at least gene or operon selected from the group consisting of a ldhA gene represented by SEQ ID NO: 5, a frdABCD operon represented by SEQ ID NOs: 6 to 9, an ackA-pta operon represented by SEQ ID NOs: 10 and 11, and a leuCD operon represented by SEQ ID NOs: 12 and 13 is deleted.

6. The strain of claim 4, wherein the strain is a strain from which a ldhA gene represented by SEQ ID NO: 5, a frdABCD operon represented by SEQ ID NOs: 6 to 9, an ackA-pta operon represented by SEQ ID NOs: 10 and 11, and a leuCD operon represented by SEQ ID NOs: 12 and 13 are deleted.

7. A method for producing citramalate comprising culturing the strain of claim 1.

8. A method for producing citramalate comprising culturing the strain of claim 2.

9. A method for producing citramalate comprising culturing the strain of claim 3.

10. A method for producing citramalate comprising culturing the strain of claim 4.

11. A method for producing citramalate comprising culturing the strain of claim 5.

12. A method for producing citramalate comprising culturing the strain of claim 6.

13. The strain of claim 7, wherein the carbon source of the medium includes alginate and mannitol.

14. The strain of claim 7, wherein the carbon source of the medium is brown microalgae.


SEQUENCE LISTING (13 sequences)

SEQ ID NO: 1 - DNA, 1119 bp, synthetic construct
  source 1..1119: mol_type = other DNA
atgatggttcgcatttttgacaccactctgcgcgatggggaacagactcccggtgttagtttaacacctaatgacaagttagaaatcgcaaagaagcttgacgaacttggtgtagatgttatcgaggcgggttcagcagtaacgtcaaaaggcgagcgcgagggaatcaaattgatcactaaggagggcctgaacgctgagatttgcagttttgtacgcgcgcttcccgtcgatattgatgcagctttggaatgtgatgttgattcggttcatttggttgtcccaacatcgccgattcacatgaagtataagctgcgcaagaccgaggacgaagttttagtcacagctctgaaggccgtggaatatgctaaggaacagggattaatcgtggagttatccgcagaggacgcaacacgctcagatgttaacttcttaatcaaacttttcaatgagggtgagaaagttggagcggatcgtgtatgtgtttgcgacaccgtcggagtcttgaccccgcagaagagccaagagttatttaaaaaaattacagagaatgttaacttgcccgtttcagtacattgccataatgactttggtatggcaacggctaacgcttgttcagcagtactgggtggcgcagtacagtgtcacgtcacggttaatggcatcggcgaacgcgctggcaacgcctcccttgaggaggtcgtagccgcatccaaaattttgtacggatatgataccaagatcaagatggaaaagctttatgaggtctctcgtatcgtatcgcgcttaatgaaactgcccgtgcctcctaacaaggctattgttggtgataatgcgtttgcccatgaggcagggattcatgttgatggcttgatcaaaaatactgaaacgtatgagcccattaaacctgaaatggttgggaatcgccgccgtatcatcttaggtaaacattccggtcgcaaagcactgaaatacaagttggatttgatggggatcaacgtgtccgacgagcaactgaacaagatctatgagcgtgtcaaggagtttggagatttaggcaagtacatctcggatgccgaccttttggctatcgttcgtgaggtaactggaaagttgtaa

SEQ ID NO: 2 - DNA, 1535 bp, Vibrio sp.
  source 1..1535: mol_type = genomic DNA
catggctcagattgaacgctggcggcaggcctaacacatgcaagtcgagcggaaacgagttaactgaaccttcgggggacgttaacggcgtcgagcggcggacgggtgagtaatgcctaggaaattgccctgatgtgggggataaccattggaaacgatggctaataccgcatgatgcctacgggccaaagagggggaccttcgggcctctcgcgtcaggatatgcctaggtgggattagctagttggtgaggtaagggctcaccaaggcgacgatccctagctggtctgagaggatgatcagccacactggaactgagacacggtccagactcctacgggaggcagcagtggggaatattgcacaatgggcgcaagcctgatgcagccatgccgcgtgtgtgaagaaggccttcgggttgtaaagcactttcagtcgtgaggaaggtttatacgttaatagcgtatagatttgacgttagcgacagaagaagcaccggctaactccgtgccagcagccgcggtaatacggagggtgcgagcgttaatcggaattactgggcgtaaagcgcatgcaggtggtttgttaagtcagatgtgaaagcccggggctcaacctcggaatagcatttgaaactggcagactagagtactgtagaggggggtagaatttcaggtgtagcggtgaaatgcgtagagatctgaaggaataccggtggcgaaggcggccccctggacagatactgacactcagatgcgaaagcgtggggagcaaacaggattagataccctggtagtccacgccgtaaacgatgtctacttggaggttgtggccttgagccgtggctttcggagctaacgcgttaagtagaccgcctggggagtacggtcgcaagattaaaactcaaatgaattgacgggggcccgcacaagcggtggagcatgtggtttaattcgatgcaacgcgaagaaccttacctactcttgacatccagagaacttttcagagatgaattggtgccttcgggaactctgagacaggtgctgcatggctgtcgtcagctcgtgttgtgaaatgttgggttaagtcccgcaacgagcgcaacccttatccttgtttgccagcgagtaatgtcgggaactccagggagactgccggtgataaaccggaggaaggtggggacgacgtcaagtcatcatggcccttacgagtagggctacacacgtgctacaatggcgcatacagagggcggccaacttgcgaaagtgagcgaatcccaaaaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggctgcaaaagaagtaggtagtttaaccttcgggggacgcttaccactttgtggttcatgactggggtgaagtcgtaacaaggtagcgctaggggaacctggcgctggatcacctcctta

SEQ ID NO: 3 - DNA, 29 bp, synthetic construct
  source 1..29: mol_type = other DNA
ttgacaattaatcatcggctcgtataatg

SEQ ID NO: 4 - DNA, 25 bp, synthetic construct
  source 1..25: mol_type = other DNA
aaacaaaaaaaaaggagcatcctca

SEQ ID NO: 5 - DNA, 873 bp, Vibrio sp.
  source 1..873: mol_type = genomic DNA
atggctcacggctgcgaagtcgtctgtgcatttgttaacgatgatctgtcagaacccgttttaaaacagctatctcagggcgggactaagcttatagcgatgcgttgtgcgggatttgataaggtcgaccaacaagcggctaagaagctaggcttgcaagtagtacatgtgcccgcctattcacctgaagcggttgccgaacatacggttggtatgatgatgtgtctaaaccgtcgactacacaaagcctatcagcgaacccgggatgcgaatttctctctggaaggtttagttggcttcaacttttttggtaagactgtaggggtaataggtacgggaaaaatcggcattgctgcgatgagaatttttaagggattaggcatggaaattctttgccatgatccttacgaaaacccactggcaatagagatgggcgcacgttactgctctcttgaagatatatacgccaatgcggatattattactttgcattgcccgatgagtaaagaaaactaccacctcctcaatgccgactcattttcgaaaatgaaagatggggtgatgatcattaatacgagtcgtggagagctattggattctgtagcagcaatcgaagcgctaaaacaaggcagaatcggctcactaggcttggatgtgtacgacaatgaaaaagagttgttcttccaggataaatcaaacgacattatcgtagacgatgttttccgccgactgtcggcatgtcataacgtgctgttcacgggtcatcaagctttcttaactcacgaagcgctcaacaacatcgcgtcagtgacactaaataacgtagaagtattcttctctgggcaagtttcaggcaatgaactgatcaactaa

SEQ ID NO: 6 - DNA, 1806 bp, Vibrio sp.
  source 1..1806: mol_type = genomic DNA
gtgcaaattatcaccacagatatcgcagtcatcggcgctggcggcgctggtcttcgtactgctattgcagcggcagaggcaaacccagatttagaagtcgctctgatttctaaagtttatcctatgcgctcacacacggtcgcagcggagggtggctcagcagcagttatcaaggatgaagatagcttagataaccacttcaacgatacggttggcggtggcgactggctatgtgaacaggacgtcgttgaatattttgtagaaaacgcaacccgcgaaatgatccaaatggaacaatggggttgtccttggagccgtaaagagaacggggaagttaacgttcgccgctttggcggtatgaaggttgaacgaacttggtttgcagcggataaaaccggcttccatatgcttcacaccctattccaaacttcaatgaagtacagcaacatcaaacgttttgatgagtactttgtgttggatctgcttgttgacgatggtgaagtacaaggtctgatcgccattcacatgtctgaaggtgagttggtcaccatcaaagcgaaatctgttgtgcttgcgactggtggcgcgggacgtgtttaccactgtaacaccaatggcggtatcgtaacgggcgatggcatggcgatggcttaccgtcacggtgttccgctacgtgatatggaatttgttcaataccaccctactggcctaccaggtactggtatcctaatgacagaaggttgtcgtggtgaaggtggtattattgtcaacaaaaacggctaccgctacctacaagactacggcatgggccctgaaactccagtgggtcaaccgaaaaacaaatacatggaactgggtccacgtgacaaagtttctcaagctttctggcatgagcaacagaaaggcaacaccatcaaacacccactgggtgacgtggtgcacctagaccttcgccaccttggtgaagagtacctacaagaacgtttacctttcatctgtgagctagcaaaagcttacgtgaacgttgatcctgcaaaagaaccaattccaattcgtccgaccgtgcactacaccatgggcggcatcgaaactgatggtggctgtgagactcgcgttaaaggtctattcgcagttggtgagtgtgcgtcagttggtctgcatggtgcgaaccgtcttggctctaactctctggctgagttcgtggtatttggccgagttgcgggtgaaaacgcagtgaaacgtgcagcagaattcaaaggctggaacgacaatgctatcgcagctcaagtgaaagctgttgaagaacgcattgccagcttaatgaaccaagaaggcgatgaaaactgggcagacatccgtaccgaaatgggccacaccatggaagcgggttgtggcatctaccgccaagaagatctgatgcaagcaaccatcgataagatcacggaacttaaacaacgttacaaacgcattagcatcaaagacaaaggcaaagtgttcaacactgaccttctttacgcaatcgaagtcggttacggcctagaagtggcagaagcgatggttcactctgcaatcctgcgcaaagaatctcgcggtgcacaccaacgtctcgatgatggctgcactgaacgtgacgacgtgaacttcctgaaacactcacttgctttctatcaaccagacgcagcgcctagcatcgactacagcaatgtaaccattactaagtctcagcctaaagcgcgtctatacggtgaagctgcagaaaaagccgcagcagaagaagcagcgaagaacgcagaggagcaagcataa

SEQ ID NO: 7 - DNA, 747 bp, Vibrio sp.
  source 1..747: mol_type = genomic DNA
atgtcagcaaaccgcatccagaaagtagacattctgcgttatgacccagaaaaagacgcagaaccgcacttacaaactttcgaagtaccattcgatgaaactatgtctgtgctcgacgcgattggttacatcaaagataacctagacaaagacttatcttaccgttggtcttgtcgtatggcgatctgtggctcatgcggcatcatggttaatggtgtgcctaagctagcttgtaagagcttcttacgtgactaccaaaatggtctgaaaatcgagccattagcgaatttcccgattgagaaagacttgatcgttgatatgacgccatttatcgagcgtcttgaagcgatcaaaccttacatcattggtaacgaccgtaaacctgaagacggcacaaacttgcaaacgccagagcaaatggcgaagtacaagcagtttgctggttgtatcaactgtggtctgtgttacgcagcgtgtcctcagttcggtctcaacccagagttcatcggcccggcagcgctaacattggcgcaccgttacaacttagacagtcgtgataacggtaaagctgagcgtatgaagctgattaacggtgagaatggcgcctggggttgtacgtttgtaggttactgttctgaggtttgtccaaagagcgttgaccctgcagcagcagtaaaccaaggcaaagtagagtcttctatggacttcgtaattgcgatgctgaaacctcaggaggcagaaggatga

SEQ ID NO: 8 - DNA, 384 bp, Vibrio sp.
  source 1..384: mol_type = genomic DNA
atgagtaaccgtaaaccttacgttcgtgaagtaaaacgcacttggtggaaggaccatcctttctaccgcttctacatgttacgtgaagcgacggtactgccactgattctattcaccatcttcctgactttcggcctgggttcactagtgaaagggcctgaagcttggcaaggctggttagagttcatggcaaacccaatcgtagtcgcgatcaacatcgttgcgctacttggaagcctgttccacgcacaaaccttcttcagcatgatgccacaggtgatgccaattcgcctaaaaggcaaacctgtgggtaagaatatcatcgtactgactcagtgggcagcggtcgcgtttatctcactgatcgttctcatcgtggtgtaa

SEQ ID NO: 9 - DNA, 378 bp, Vibrio sp.
  source 1..378: mol_type = genomic DNA
atgaaaccaaattatagtgtaaacacagcaccaaaacgttcagatgagccaatctggtggggactgtttggtgcaggcggtacctggtttgcgatgatcactcctatcaccgtacttgtacttggtatcctcgttccactgggcgtgattgatgcagacgccatgagctacgagcgagtatctgaattcgcgaccagtatcattggtgcgctatttatcatcggtacactagcgctgccaatgtggcatgcaatgcaccgtgttcaccacggcatgcacgaccttaagttccacactggtgtggtgggaaaagtggcatgctatgcgttcgctggccttatcagtgcgctatcagttatctttatcttcatgatttaa

SEQ ID NO: 10 - DNA, 1197 bp, Vibrio sp.
  source 1..1197: mol_type = genomic DNA
atgtctaagctagttttagttttaaactgcggtagttcttctcttaaatttgccgtagttgatgccgagaacggcgacgagcacttatcaggtcttgctgagtgtcttcaccttcctgaagctcgtatcaagtggaaacttgatggcaagcacgaagctcaactaggtaacggtgcagctcacgaagaagcactagcgttcatggtagaaactattcttgcttctaagccagagctttctgaaaacctagcagctatcggtcaccgtgtagtacacggcggcgagaagttcactcagtctgctcttatcactgatgacgtactgaaaggtatcgaagactgtgcaacactagcaccacttcataacccagcacacatcatcggtatcaaagcagctcaaaaatctttcccagcactgaaaaacgttgctgtgttcgacactgcattccaccaaacaatgccagaagagtcttacctatacgctcttccatacaacctgtacaaagagcacggcatccgtcgctacggtatgcacggtacttcacacctattcatcactcgtgaagttgcaggtctactgaacaagccagttgaagaagtaaacatcatcaactgtcacctaggtaacggcgcgtctgtatgtgcagttaaaaacggtaaatcagtagatacttctatgggtctgactcctctagaaggtctggtaatgggtactcgttgtggtgacatcgatcctgcgatcatcttccacctacacgacgcactaggctactctgttgagaaaatcaacacgatgctaactaaagagtctggcctacttggtctgactgaagtgacttctgactgtcgtttcgttgaagacaactacggtgagaaagaagaagcaacgcgtgcaatggacgtgttctgtcaccgtctagctaaatacgtagcgggttacactgcatcaatggacggccgtctagatgcgatcactttcactggcggtatcggcgagaactctggccctatccgtgaaatggttctaaaccgtctaggtatcttcggtatcgaagtagacagtgaagcaaaccttaaagcacgtttcggcggcgaaggcactatcactactgcgaacagccgtatcccagcaatggtaatctctactaacgaagagcttgttattgctgaagacactgcacgcctagcaggtctttaa

SEQ ID NO: 11 - DNA, 2145 bp, Vibrio sp.
  source 1..2145: mol_type = genomic DNA
atgtctcgtactattatgcttatccctgcaagtgctggtgttggtttaactagcgtaagcatgggtgttcttcgcgctatggagcgcaaaggcgttaaagtttctttctacaaacctatttgtcagccacgcagtggcggcgatcagccggatctaacttcaactatcgttggccacaacagcgatatgaagatcggcgaacctatggcgatgtctgttgctgaaagcctgatcggtaacgacaacatggatgagctgctagaaaccatcgttgagcgttacaaccaaatcaataaagatgcagatgtaactctaatcgaaggtctggtaccaactcgtaagcacccattcgctaaccaagtgaacgcggaaatcgcagcaacacttggtgctgaaattgttctagtggcaacgcctggcacggataacccagctcagcttaaagagcgcatcgaagtagcatgttctaacttcggcggtactaagaacaaaaacatctctggtgttatcatcaacaagctaaacgcacctgttgatgaagctggccgtactcgccctgacctgtctgagatctttgacgatgcagacagcgcgaagcaaaacgaaatgaaagtgatggagatcttcaacacttctccaattcgtgtactaggttgcgtgccatggagcatcgacctgattgcaactcgtgctatcgacatggcgaaacacctgaatgctgatatcatcaacaaaggtgacatcaacactcgtcgcatcaagagcatcacgttctgtgcgcgttctctgccaaacatgattgagcacttcaaaccaggttctctacttgttacttctgcagaccgtccagacgtaatcgttgcggcttctctagcggcaatgaacggtgttgacatcggtgctgtactgctaaccggcggttacgacatccctcgcgaaattgaaggtctatgtaagccagcattcgaaactggtctacctatcttcaaagctcaaggcaacacttggcagacttctctaaatctacagagcttcagcattgaagtacctgcagacgacaaagagcgtatcgaattcattaacgagcacgttgctggtcacatcgacggtaactggatcgagtcaatgactgaaggtactcagaagtctcgtcgtctaagcccaccagcattccgttaccagctaactgaacttgctcgtagagccggcaagcgcatcgttcttccagaaggtgatgagccacgtaccgtgaaagctgctgctatctgtgctgagcgcggcattgcagaatgtgtgcttctgggtaacccagcagaaatccgtcgcgtagctgcacaacaaggtgttgagctaggcgctggcgttcaaatcatcgacgctgatgctatccgtgaaaactacgttgctcgcctggttgagctacgtggtgcgaaaggtatgactgaagttgtagctcgtgagaaactacaagactcagtattcctgggcacaatgatgcttgagaacaacgaagttgatggcctggtttctggtgctgttcacactacggcgaacactatcgttcctccgttccaaatcatcaagactgcacctaatgcgtctatcgtttcttcagtattcttcatgcttctgcctgatcaagtactggtttacggtgactgtgcaatcaacccagatccaacggctgaacagcttgctgaaatcgctatccaatcagcagattctgctgctgcattcggtatcgacccacgcgttgcaatgatttcttactctactggtgaatctggtaagggtgcagacgttgataaagtacgtgaagcgacgaaactagctcaagagaaacgtcctgatcttatcatcgacggtcctctacagtacgacgcggctatcatggaaaacgttgcggcttctaaagcgccaaactctccagtagcaggtaaagcgacagtattcgtattcccagacctaaacactggtaacacgacttacaaagcagtacagcgttcagcagatctagtatctatcggtccaatgctgcaaggtatgcgcaagcctgtaaacgacctgtctcgtggcgcgctagtagacgacatcgtgtacacaatcgctctaacagcaatccaagcaactcaagaacagaagtaa

SEQ ID NO: 12 - DNA, 1401 bp, Vibrio sp.
  source 1..1401: mol_type = genomic DNA
atgggcaaaacactttacgagaaggtctacgacgcacacgttgctgtcgcagctgaaggggaaaacccgattctttatattgatcgtcacttggtacacgaagttacctctccacaggcttttgatggcttacgtgaaaagggccgtcaagtgcgtcaggtaagcaaaacgttcgcaacgatggaccacaacgtttcgaccacaaccaaagacatcaacgcttctggcgagatggctcgtatccagatggaaacgctatcgaaaaactgtgaagaattcggtgtaaccctttacgacctaaaccacaaataccaaggcattgtccacgttatgggccctgagctaggtattactctgccgggcatgaccattgtttgcggtgactctcatacagcaacacacggtgcgttcggttcactggctttcggtatcggtacatctgaagttgaacacgtactagcgactcaaacgctaaaacaagcacgtgcgaaaacaatgaaaatcgaagtgaaaggtaaagtcgctccgggcattacagcaaaagatatcgtactagcgatcatcggtaaaacgacagctgcaggcggtactggctacgtggttgagttctgcggtgaagcgatcacggacctttcaatggaaggccgtatgacagtatgtaacatggctatcgaattgggcgcgaaagcgggtctgattgcaccggatgaaaccacgttcgaatacatcaaaggccgtaaattctcaccacaaggtgcagattttgacgcggcggttgaatactggaaaacactaaaaactgacgatgatgctgaatttgatgccgtagtaacgctaaacgcggcggacatcaaaccgcaagtaacttggggtacaaacccaggtcaggttatcgcggttgaccaaccgattccttcaccagaaagtttttcagacccagtggaaaaagcatcggcagcaaaagcgctggcttacatgggactagaagcgggtaaatcactgtcagactaccctgttgataaagtgtttgttggttcttgtactaactcgcgtattgaagacatgcgcgcagcagcagaagtggctaaaggccgcaaagtcgcatcgcacgttcaagcattaattgttcctggctctgagcaagtgaaggcacaagcggaagcagaaggcttggatgtcatctttaaagaagcgggctttgaatggcgtctaccaggctgttcaatgtgtctggcgatgaacaacgaccgcctaggtcctcaggagcgttgtgcatcgacttcaaaccgtaactttgaaggtcgtcaaggccgtgatggtcgtacccacttggttagtcctgcaatggcggcggcggcggctatcgctggtcactttgttgatattcgtgaactagattaa

SEQ ID NO: 13 - DNA, 603 bp, Vibrio sp.
  source 1..603: mol_type = genomic DNA
atgtcaggttttaaacaacacaaaggcttagttgttcctctagatgcagcgaacgttgatactgatgccatcataccaaagcagttcttacaaaaggtgtctcgcctgggtttcggtaaacacctattccatgactggcgcttccttgatgatgctggtgaacaaccaaaccctgagtttgtgatgaacgcaccacgttaccaaggtgcttctatcctacttgcgcgtgaaaacttcggttgtggctcttctcgtgagcacgcaccttgggcgttagccgattacggcattaaagcgatgatcgcaccaagctttgcagatatcttctatggcaactcaatcaacaaccaaatggtgccagtgcgtctgactgagcaagaggttgatgaactgttccaatacgttgaagcgaatgaaggtgcacagattgaagtggatcttgaagcgaacaaagtacgtgccaatggcaaagagtacgatttcgaaatcgatgagttccgtcgccactgtctactgaatggcttggacaacatcggtttgacgctacagcacgaagataaaattgcggaatatgaagcgaatatcccaagcttcctgcgctaa